(19) United States
(12) Patent Application Publication
(10) Pub. No.: US 2023/0374460 A1
(43) Pub. Date: Nov. 23, 2023

(54) Methods for Cardiac Differentiation of Human Induced Pluripotent Stem Cells

(71) Applicant: The Trustees of Columbia University in the City of New York, New York, NY (US)

(72) Inventors: Masayuki Yazawa, New York, NY (US); LouJin Song, Cambridge, MA (US)

(21) Appl. No.: 18/362,365

(22) Filed: Jul. 31, 2023

Related U.S. Application Data
(63) Continuation of application No. 15/755,812, filed on Feb. 27, 2018, now abandoned.
(60) Provisional application No. 62/212,195, filed on Aug. 31, 2015.

(51) Int. Cl. C12N 5/077; C12N 5/074

(52) U.S. Cl. CPC C12N 5/0657; C12N 5/0696; C12N 2501/999; C12N 2500/90; C12N 2506/1307; C12N 2506/45; C12N 2506/094; C12N 2500/99; C12N 2501/33; C12N 2501/727; C12N 2500/02; C12N 2501/415

(57) ABSTRACT

The present invention relates to monolayer cardiac differentiation techniques utilizing defined conditions providing feeder-free monolayer culture systems, serum-based or serum free, and applicable to both healthy control and patient derived stem cells.

### CROSS-REFERENCE TO RELATED APPLICATIONS

The present application is a continuation application of U.S. patent application Ser. No. 15/755,812, filed Feb. 27, 2018, which is a U.S. National Phase application under 35 U.S.C. § 371 of International Patent Application No. PCT/US2016/049576, filed Aug. 31, 2016, and claims priority to U.S. Provisional Patent Application No. 62/212,195 filed Aug. 31, 2015, each of which is incorporated herein by reference in its entirety. The International Application was published on Mar. 9, 2017, as International Publication No. WO 2017/040579 A1.

### GRANT INFORMATION

This invention was made with government support under HL 111345 awarded by the National Institutes of Health. The United States Government has certain rights in the invention.

### FIELD OF THE INVENTION

The present invention relates to monolayer cardiac differentiation techniques utilizing defined conditions providing feeder-free monolayer culture systems, serum-based or serum free, and applicable to both healthy control and patient derived stem cells.

### BACKGROUND OF THE INVENTION

Generating cardiovascular cells from pluripotent stem cells holds great promise for cardiovascular research and therapy. However, pluripotent stem cell differentiation into cardiac cells is inefficient and results in heterogeneous cultures, limiting the usefulness of this approach. Pluripotent stem cells, such as human embryonic stem (hES) cells and induced pluripotent stem (iPS) cells, can perpetually proliferate and differentiate into derivatives of all three embryonic germ layers (Thomson et al., Science 282:1145 (1998); Odorico et al., Stem Cells 19:193 (2001); Yu et al., Science 318(5858):1917 (2007)). Differentiation of pluripotent stem cell cultures can occur spontaneously, which results in a seemingly random variety of cells (Watt and Hogan, Science 287:1427 (2000)). The earliest methods of pluripotent stem cell differentiation included allowing stem cell aggregates to spontaneously differentiate and form embryoid bodies (EBs) that contain derivatives of the three primary germ layers including in some cases cardiomyocytes.

Generating cardiomyocytes from pluripotent stem cells through EB formation is inefficient, however, as only few percent of the developing cells become cardiomyocytes. Efficient, reproducible methods for differentiating human pluripotent stem cells into cardiovascular cell lineage remain to be elucidated.

More recently, researchers attempted to improve efficiency by differentiating hES cells into cardiomyocytes without EB formation by sequentially applying growth factors or small molecules to mimic cardiac development. Soluble factors important for embryonic cardiac development include Activin A, BMP4, nodal, Wnt agonists and antagonists, bFGF and other molecules (Conlon et al., Development 120(7):1919 (1994); Lough et al., Dev. Biol. 178(1):198 (1996); Mima et al., PNAS 92(2):467 (1995); Zaffran and Frasch, Circ. Res. 91 (6), 457 (2002)). Various combinations of these factors have been tested in cardiac differentiation protocols. Treatment with Activin A and BMP4 promoted cardiogenesis of H7 ES cells grown as monolayers (Laflamme et al., Nat. Biotechnol. 25 (9):1015 (2007)). Other protocols employed EB-based differentiation and enhanced cardiogenesis by using various combinations of BMP4, Activin A, bFGF, VEGF, and dickkopf homolog 1 (DKK1) (Yang et al., Nature 453 (7194):524 (2008)). The latter protocols were performed using H1 and HES2 human ES cell lines and have not been demonstrated to work with other ES cell lines or iPS cell lines.

However, these recombinant proteins are expensive and unstable and have not been applicable for patient-derived iPS cells that are more fragile than ES cell lines or healthy control iPS cells. Thus, there is a need for reproducible, cost-effective and efficient methods for differentiating human iPS cells, especially patient-specific iPS cells, into cardiomyocytes in vitro.

### BRIEF DESCRIPTION OF THE FIGURES

FIGS. 1A-H: Optimized monolayer differentiation into cardiomyocytes. (FIG. 1A) Time schedule of monolayer differentiation into beating cardiomyocytes (CMs) using the serum containing protocol. (FIG. 1B) Gene expression of cardiac myofilaments, ACTN2, MYH7 and TNNT2, in control and LQTS cells at day 4 and day 19 using qPCR, normalized to GAPDH expression. ** P<0.01 with Student t-test between day 4 and day 19. (FIG. 1C) FACS analysis using sarcomere alpha-actinin (SA) antibody in control and LQTS differentiating cells at day 4 and day 19. ** P<0.01 with Student's t-test between day 4 and day 19. The cell samples tested for FACS were identical with ones used for qPCR (mean±s.e.m. n=8-14, in 4 controls and 3 patient lines from two LQTS patients). (FIG. 1D) Imaging of calcium transients in control single cardiomyocyte using confocal microscope. Line scan image (top, black/white) and calcium transients (bottom) are shown (n=102). (FIG. 1E) Time schedule of monolayer differentiation into beating CMs using serum-free protocol. (FIG. 1F) FACS analysis using SA antibody in differentiating cells at day 19 with serum-free protocols. n.s., there is no significant difference between IMDM and RPMI media supplemented with B27 at day 19 with Student's t-test. n.d., not determined: the cells prepared in RPMI media with albumin and ascorbic acid (RPMI AL/VC) could be not fully dissociated with trypsin at day 19, resulting in forming cell aggregates. (FIG. 1G) The number of SA-positive cells using serum-free protocols in a well of 6-well plate. ** P<0.01 with Student's t-test between IMDM and RPMI supplemented with B27 (mean±s.e.m.). (FIG. 1H) The gene expression of ACTN2 at day 19, normalized to GAPDH. One-way ANOVA was used (mean±s.e.m. n=14-19, two controls and a patient line). n/a, no expression of ACTN2 at day 19 (n=11). The cell samples tested for qPCR were the same as the ones used for FACS analyses.

FIGS. 2A-C: Dual optical recording of action potentials and calcium handling in single cardiomyocytes. (FIG. 2A) Representative traces of voltage (top, black) and calcium ions (bottom, gray) in control single cardiomyocyte. (FIG. 2B) Dual optical recording of action potential and calcium handling in LQTS CMs before, during and after treatment with 5 μM roscovitine (Ros). Ros prevented irregular calcium handling and electrical activities observed in LQTS CMs. (FIG. 2C) Roscovitine rescued prolonged action potentials in LQTS cardiomyocytes. ** P<0.01 with Student's t-test (n=12, 4 lines from two LQTS patients). Y-axis, ΔF/F0 for R-GECO1 and −ΔF/F0 for ArcLight.

FIG. 3: Using GSK3 and Wnt Inhibitors to differentiate IPSCs into spontaneously beating cardiomyocytes. Comparison of method (i) using embryoid bodies (EBs) formation to our optimized protocol (ii) using monolayer differentiation with glycogen synthase kinase 3 (GSK3) inhibitors CHIR99021 (CHIR) and BIO and Wnt inhibitor IWP in DMEM/F-12 with FBS, two established monolayer protocols (iii) using activin A (AA, 100 ng/ml) and BMP4 (10 ng/ml) in RPMI media with B27 (w/o insulin, −I) and then B27 (+insulin, +I) (Laflamme et al., 2007) and (iv) CHIR and IWP in RPMI media with B27(−I) and then B27(+I) (Lian. et al., 2013). The optimization to develop the protocol (ii) was based on the established protocol (i) using EB formation because all tested control and patient iPSC lines could generate beating cardiomyocytes using the protocol (i). The recently reported protocol using minimized factors, albumin (AL) and ascorbic acid (VC) was reproducible to obtain beating cardiomyocytes (v). However, with protocol (v), dissociation with trypsin into single cells was more difficult compared to the other protocols. An optimized protocol using IMDM in this study is reproducible and efficient in multiple lines to obtain higher yield of cardiomyocytes that were positive to cardiac myofilaments (vi). Even using an established protocol (iii) with IWP (2 μM) and B27 (−I) as optional induction, a control iPSC line that had been cultured in E8 media could not be differentiated into any beating cardiac cells (the expression of myosin heavy chain was not detected. N.D. in culture at day 33) while our protocol (ii) provided beating cells at day 11 reproducibly. Using protocol (iii), after changing E8 media to differentiating media, many dead cells were observed and no spontaneously beating cardiomyocytes were found. Using protocol (iv), only around the edge of the well, small aggregates of control beating cells were found at day 11 while no control beating cells were around the center. No LQTS beating cells were found anywhere on either center or edge of wells. According to our experience, Protocol (ii) was not as sensitive to initial iPSC density as protocol (iv). With ˜90-95% confluent of control and patient iPSC lines, Protocol (iv) could provide relatively more cardiomyocytes. However, approximately 50% of differentiation using this protocol (iv, 12 out of 25 wells from four trials) still failed to provide cell samples for FACS and qPCR while the results of protocols (ii), (v), (vi) were consistent using ˜90-95% confluent of control and patient iPSCs. In our experiments, only when beating cells were observed in the center of wells using protocol (iv) by day 19, the cell samples were used for FACS and qPCR. we also found that continuous hypoxia reduced differentiation efficiency using protocol (ii) to beating cells as well as temporal hypoxia in mesodermal differentiation phase (day 1-4) and then normoxia (day 5˜, Table 1).

FIGS. 4A-C: Efficient reprogramming of patient skin fibroblasts using single lipofection. (FIG. 4A) Outline of the reprogramming of skin fibroblasts into iPSCs using lipofection with Lipofectamine LTX in 24-well plate. (FIG. 4B) The number of iPSC-like colony generated from human skin fibroblast and hair keratinocytes (FK) plated in 24-well plate scale (n=4-8 in two independent experiments). FK*, no iPSC-like colonies (n/a) were found in case of keratinocyte media usage at day 2-6. (FIG. 4C) Expression of pluripotent marker Nanog and house-keeping gene GAPDH in iPSC lines, H9 line (positive control, P. Con), untreated patient fibroblasts (Fib, negative control, N. Con) and transfected fibroblasts using the episomal vectors (collected at day 5 after lipofection).

FIGS. 5A-J: Dual optical recording using ArcLight (black trace) and R-GECO1 (gray trace) in control and patient cardiomyocytes. (FIG. 5A) Dynamic changes in cellular phenotypes were observed in LQTS cardiomyoctes at day 23-40. (FIG. 5B) Roscovitine test using dual optical recording of action potential (black) and calcium handling (gray) using ArcLight and R-GECO1 in monolayer of LQTS cardiomyocytes generated using serum-containing protocol (FIG. 1A). Roscovitine shortened action potential (FIG. 5C, APD09, 90% from peak) and increased beating rate as well as the number of spontaneous calcium transients (FIG. 5D, n=31, 3 LQTS lines from two patients). (FIG. 5E) Dual optical recording of action potential (black) and calcium handling (gray) in a monolayer of control (Ctrl) and LQTS cardiomyocytes generated using serum-free protocol (FIG. 1E) before and after treatment with 5 μM roscovitine (10 min). There was no effect of roscovitine on APD90 (FIG. 5F) and the number of spontaneous calcium transients (FIG. 5G) in Ctrl cardiomyocytes (n=9 in a control line) while positive effects were observed on APD90 (FIG. 5H) and the number of spontaneous calcium transients (FIG. 5I) in LQTS cardiomyocytes prepared under serum-free condition (n=22, 3 LQTS lines from two patients). (FIG. 5J) There were no obvious phenotypes in atrial-like (left) and nodal-like cardiomyocytes (right) generated from LQTS iPSC (bottom) compared to control cells (top). Y-axis, ΔF/F0 for R-GECO1 and −ΔF/F0 for ArcLight. ** P<0.01 with Student's t-test.

FIGS. 6A-B: Quantification of the cell size from the Immunocytochemistry images (using anti-alpha-actinin antibody) of control (Ctrl, left) and LQTS cardiomyocytes that were prepared using serum-containing protocol (FIG. 1A) or serum-free protocol (FIG. 1E). While there was no significant increase under serum-containing culture (FIG. 6A), the cell size significantly increased in LQTS cardiomyocytes under serum-free condition (FIG. 6B). *P<0.0001 with Student's t-test (mean±s.d., 4 controls and 3 LQTS lines).

### SUMMARY OF THE INVENTION

The present invention relates to monolayer cardiac differentiation techniques utilizing defined conditions providing feeder-free monolayer culture systems, serum-based or serum free, and applicable to both healthy control and patient derived stem cells. In certain embodiments, the methods include administering CHIR and BIO (which are types of GSK3 inhibitors), along with IWP-3 (a Wnt inhibitor) to the cultures. In additional embodiments, the cells can be infected with lentiviruses containing genetically encoded fluorescent indicators R-GECO1 and ArcLight, which allow for noninvasive screening methods for the phenotypic responses to pharmaceutical libraries for up to 90 days after initial infection.

In additional embodiments, the methods include a method for inducing cardiac differentiation of a pluripotent stem cell, comprising: 1) culturing a pluripotent stem cell in a medium containing at least two GSK3 inhibitors and 2) culturing a cell from step 1) in a medium containing one or more WNT signaling inhibitors.

In certain embodiments, the two GSK3 inhibitors comprise CHIR and BIO. In additional embodiments, the WNT signaling inhibitor comprises IWP-3. In certain embodiments, the method is used to prepare a cardiomyocyte. In additional embodiments, the media in step 1) and in step 2) do not comprise serum.

In additional embodiments, the media in step 1) and in step 2) do not comprise a protein other than albumin.

In additional embodiments, the method further comprises infecting the pluripotent stem cell with a construct comprising one or more fluorescent indicators.

In additional embodiments, the fluorescent indicators comprise R-GECO1 and ArcLight.

In yet additional embodiments, the invention relates to a method for reprogramming or producing a pluripotent stem cell, comprising transfecting in a single step a fibroblast or a keratinocyte with episomal vectors pCXLE-hOCT3/4-shp53-F, pCXLE-hSK, and pCXLE-hUL, wherein the transfection is performed using cationic lipid. The vectors were used to induce OCT3/4, SOX2, KLF4, L-MYC, LIN28 episomal expression and reduce p53 expression in somatic cells for reprogramming without integration. Thus, any alternative plasmids that could achieve the same functions in combination with the protocol could be used for reprogramming.

In additional embodiments the cationic lipid is Lipofectamine 2000 or Lipofectamine LTX. In further embodiments, alternative lipofection reagents could be used, but these may lead to a reduced reprogramming efficiency.

In yet additional embodiments, the invention relates to a composition comprising cardiac differentiated stem cells produced by the methods described herein.

In certain embodiments, the cells exhibit at least one Timothy Syndrome phenotype selected from the group consisting of slower, irregular contractions and abnormal calcium handling.

In certain embodiments, the pluripotent stem cell is derived from a healthy control cell.

In certain embodiments, the pluripotent stem cell is derived from a patient derived cell.

In yet additional embodiments, the invention relates to a kit for promoting cardiac differentiation comprising at least two GSK3 inhibitors and one or more WNT signaling inhibitors and the composition comprising cardiac differentiated stem cells produced by the methods described herein.

In yet additional embodiments, the invention relates to a kit for promoting cardiac differentiation comprising at least two GSK3 inhibitors and one or more WNT signaling inhibitors, wherein the kit is used for any of the methods described herein.

As described herein, the methods and kits are suitable for the large-scale, reproducible production of human cardiomyocytes, and work well in control iPS cells as well as in patient-derived iPS cells.

### DETAILED DESCRIPTION

The present invention relates to cardiac differentiation techniques including monolayer cultures that exhibit homogeneous exposure to the nutrient components. In certain aspects, the culture methods encompass the application of three chemical ingredients: CHIR and BIO (which are types of GSK3 inhibitors), along with IWP-3 (a Wnt inhibitor) for feeder-free monolayer culture systems, serum-based or serum free, and applicable to both healthy control and patient derived iPS cells. In additional embodiments, the cells can be infected with lentiviruses containing genetically encoded fluorescent indicators R-GECO1 and ArcLight, which allow for noninvasive screening methods for the phenotypic responses to pharmaceutical libraries for at least up to 75 days after initial infection. The present culture methods do not rely on expensive and unstable recombinant factors such as Activin A or bone morphogenetic protein 4 (BMP4). Advantages of the present methods include suitability for the large-scale, reproducible production of human cardiomyocytes for high-through-put screening. Additionally, the present methods work well in control iPS cells as well as in patient-derived iPS cells, which are typically more fragile.

As an initial step, we developed an optimized protocol for the reprogramming of human fibroblasts and keratinocytes into pluripotency using single lipofection and the episomal vectors (pCXLE-hOCT3/4-shp53-F, pCXLE-hSK, pCXLE-hUL) in 24-well plate format. This method allowed us to generate multiple lines of integration-free and feeder-free induced pluripotent stem cells from multiple patients and controls. The iPS cell lines derived from the human skin fibroblasts were used for the following monolayer cardiac differentiation. We developed two monolayer cardiac differentiation protocols to differentiate both control iPS cells and Timothy patient specific iPS cells into cardiomyocytes. Using those monolayer differentiation methods, we generated human cardiomyocytes from the iPS cell lines derived from Timothy syndrome (TS) patients, who have a missense mutation (G406R) in CACNA1C gene encoding L-type voltage-gated calcium channel Caᵥ1.2. The TS mutation leads to ineffective channel inactivation, which cause multisystem dysfunctions including lethal arrhythmia, congenital heart defects and autism (Yazawa et al. Nature 471:230-234 (2011); Splawski, et al. Cell. 119(1): 19-31 (2004)). These Timothy syndrome cardiomyocytes derived using the present monolayer cardiac differentiation methods exhibited slower, irregular contractions and abnormal calcium handling compared to controls, these phenotypes are consistent with previous cardiomyocyte phenotypes (Yazawa et al. Nature 471:230-234 (2011)). Additionally, efficient approaches have been developed utilizing these methods for recording action potentials and calcium transients in control and patient cardiomyocytes using genetically encoded fluorescent indicators. Using the Timothy syndrome cardiomyocytes and efficient new phenotyping methods, we demonstrated that Roscovitine rescued the phenotypes in action potentials, channel inactivation and calcium handling in Timothy syndrome cardiomyocytes (Song, et al. Stem Cells Transl Med. 4(5):468-75 (2015)). Importantly, these results illustrate the applicability of these methods and cell cultures as a disease model. Further, all the new results recapitulate phenotypes demonstrated by the previous conventional electrophysiological recordings and calcium imaging with dyes (Yazawa et al. Nature 471:230-234 (2011)). Thus, these findings serve to validate that these Timothy syndrome cardiomyocytes are reliable disease models and Roscovitine is a valid lead therapeutic compound for Timothy syndrome. Thus, we moved on to optimize the lead compound Roscovitine and search for new potential therapeutic compounds with higher efficacy and a similar structure with Roscovitine. Using our efficient screening approaches, twenty Roscovitine analogs were tested with two out of the candidates rescuing the phenotypes in Timothy syndrome cardiomyocytes. The approaches using the present optimized methods and recordings will serve as useful high-throughput screening methods for testing potential therapeutics and will be suitable for determining efficacy and toxicity of potential therapeutics.

### Definitions

As used herein, the term “differentiation” as used with respect to cells in a differentiating cell system refers to the process by which cells differentiate from one cell type (e.g., a multipotent, totipotent or pluripotent differentiable cell) to another cell type such as a target differentiated cell.

As used herein, the term “induced pluripotent stem cells” commonly abbreviated as iPS cells or iPSCs, refers to a type of pluripotent stem cell artificially generated from a non-pluripotent cell, typically an adult somatic cell, or terminally differentiated cell, such as fibroblast, a hematopoietic cell, a myocyte, a neuron, an epidermal cell, or the like.

As used herein, the term “cell differentiation” refers to a process by which a less specialized cell (i.e. stem cell) develops or matures or differentiates to possess a more distinct form and/or function into a more specialized cell or differentiated cell, (i.e. cardiac cell).

As used herein, the term “GSK3 inhibitor” refers to a substance which inhibits GSK3. Specific examples of GSK3 inhibitors include Kenpaullone, 1-Azakenpaullone, CHIR99021, CHIR98014, AR-A014418, CT 99021, CT 20026, SB216763, AR-A014418 SB 415286, TDZD-8. Further exemplary GSK3 inhibitors available from Calbiochem BIO (2′ Z,3′ £)-6-Bromoindirubin-3′-oxime; BIO-Acetoxime (2′ Z,3′ E)-6-Bromoindirubin-3′-acetoxime, (5-Methyl-1H-pyrazol-3-yl)-(2-phenylquinazolin-4-yl)amine, Pyridocarbazole-cyclopenadienylruthenium complex, TDZD-8 4-Benzyl-2-methyl-1,2,4-thiadiazolidine-3,5-dione, 2-Thio(3-iodobenzyl)-5-(1-pyridyl)-[1,3,4]-oxadiazole, OTDZT 2,4-Dibenzyl-5-oxothiadiazolidine-3-thione, alpha-4-Dibromoacetophenone), AR-AO 14418 N-(4-Methoxybenzyl)-N′-(5-nitro-1,3-thiazol-2-yl)urea, 3-(1-(3-Hydroxypropyl)-1H-pyrrolo[2,3-b]pyridin-3-yl]-4-pyrazin-2-yl-pyrrole-2,5-dione, TWS1 19 pyrrolopyrimidine compound, AR-A0144-18, SB216763, and SB415286. In the method of the invention, two or more GSK3 inhibitors may be used in combination.

As used herein, the term “WNT signaling inhibitor” refers to a substance which inhibits the WNT signaling pathway. Examples of the WNT signaling inhibitor include compounds such as IWP-3, IWP-2, XAV939, and IWR-1, and proteins such as IGFBP4 and Dkk1. Preferably, the WNT signaling inhibitor used in the present disclosure is a compound. In the method of the invention, two or more WNT signaling inhibitors may be used in combination.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA) from a transcribed gene.

As used herein, “polymerase chain reaction” or “PCR” refers to a procedure or technique in which specific nucleic acid sequences, RNA and/or DNA, are amplified as described in, e.g., U.S. Pat. No. 4,683,195. Generally, sequence information from the ends of the region of interest or beyond is used to design oligonucleotide primers. These primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

### Kits

The present invention also provides kits comprising the components of the combinations of the invention in kit form. A kit of the present invention includes one or more components including, but not limited to, cardiac differentiated pluripotent stem cells, or compositions thereof, GSK3 inhibitors, WNT signaling inhibitors, or similar compositions, as discussed herein.

In one embodiment, a kit includes additional compounds/composition of the invention or a pharmaceutical composition thereof in one container (e.g., in a sterile glass or plastic vial) and a second pharmaceutical composition in another container (e.g., in a sterile glass or plastic vial).

In another embodiment of the invention, the kit comprises a combination of the invention, including one or more GSK3 inhibitors in combination with one or more WNT signaling inhibitors, optionally in combination with one or more therapeutic agent components. The components can be in separate containers, or certain components may be combined for storage and transport into a single container.

In certain embodiments, the cardiac differentiated pluripotent stem cells are control cells derived from a healthy individual (i.e., one with no apparent cardiac disease). In additionally embodiments, the cardiac differentiated pluripotent stem cells are test cells derived from a patient with a cardiac-related disease (i.e. Timothy Syndrome, or Long QT syndrome). In certain embodiments, such control or test cells can be formulated into a composition suitable and stable for transport and for passaging and utilizing for screening.

The kit can include a package insert including information concerning the pharmaceutical compositions and dosage forms in the kit. For example, the following information regarding a combination of the invention may be supplied in the insert: how supplied, proper storage conditions, references, manufacturer/distributor information and patent information.

### EXPERIMENTAL PROCEDURES

### Cell Culture

HEK 293T cells were cultured in Dulbecco's Modified Eagle Media (DMEM, high glucose, #10313-021/039) with 1% GlutaMax I and 100 unit/ml penicillin and 100 μg/ml streptomycin (P.S., all reagents from Life Technologies) and 10% fetal bovine serum (FBS, not heat-inactivated, HyClone, #SH30071.03, Lot #AYE161479, Thermo Scientific) under normoxia (20% O₂, 5% CO₂, at 37° C.) using HERAcell (Thermo Scientific) and Biosafety Cabinet Class II type A2 tissue culture hood (Labconco) for culture procedures. Human embryonic lung fibroblast IMR90 (ATCC), patient dermal fibroblasts and normal adult human dermal fibroblasts (NHDF, Life Technologies) were cultured in DMEM (high glucose as above) with 1% GlutaMax I, P.S. and 10% or 20% FBS under normoxia. Skin fibroblasts of two long QT syndrome patients (LQTS, type 8, Timothy syndrome #TS7643 and #TS9862) were transferred from Stanford University under material transfer agreement. Congenital heart disease (CHD) patient fibroblasts were obtained by taking a small piece of skin at the incision at the time of surgery following a protocol approved by the Columbia Institutional Review Board (IRB, for W.K.C.). Conventional iPSC culture on feeders was conducted on home-made CF-1 feeder cells using DMEM/F-12 (with GlutaMax I, #10565-018/042, Life Technologies) containing 20% Knockout Serum Replacement (KSR, Life Technologies), 1% MEM non-essential amino acid (Life technologies), 0.1 mM beta-mercaptoethanol (bME, Sigma-Aldrich), P.S. and 10 ng/ml bFGF (#233-FB, R & D Systems) under hypoxia (5% O₂, 5% CO₂, 90% N₂ at 37° C.) using HERAcell incubator. CF-1 feeder cells were prepared as following: mouse embryonic fibroblasts (MEF, embryonic day 14.5) were isolated from CF-1 mouse strain using standard protocol with 0.25% Trypsin and EDTA solution and DNase I solution (Stem Cell Technologies). DMEM (high glucose) with 1% GlutaMax I, P.S. and 10% FBS was used for MEF culture under normoxia. After confirming cell proliferation, the next day all MEF isolated from 4-5 embryos were plated onto five 225T culture flasks (Corning). Four days after embryo dissection, mitomycin C treatment (100 μg/ml, Sigma Aldrich) was applied and feeder stocks were prepared (total ˜1.2×10⁸ cells from five embryos). Half million of CF-1 feeder was plated in a well in 6-well plate for conventional iPSC culture.

Feeder-free iPSC lines were cultured in E8 media (Chen et al., 2011): Essential 8 basal media (prototype: A14625DJ, Lot #1350709 & #1376319; Current ones, A15169-01, Lot #1445548 & #1505926) with Essential 8 supplement (prototype, and current version A15171-01, Lot #14455555 & #1493471, Life Technologies) under hypoxia and normoxia conditions on plates or dishes (Corning) coated with Geltrex following the manufacturer's instruction (#A14133-022, Life Technologies). The iPSC lines under hypoxia and normoxia were incubated with 1 mg/ml dispase (Life Technologies) for 4-5 min at 37° C. and three times wash using E8 media (3×1 ml per well) and then add 1.5 ml E8 media supplemented with 10 μM Y27632 (CalbioChem #688000 and TOCRIS #1254/50) in case of 6-well plate. Under a stereoscopy (Nikon, SMZ745) in Purifier Horizontal Clean bench (Labconco), we picked up 3-4 uniformed (ESC-like) colonies using small needle (27G, ½ inch, BD) and then transferred to 1.5 ml tube using filter tip (GP-L200F, Rainin). The cells were gently dissociated using filtered tips (#GP-L1000F, Rainin) with 8-10 times pipetting and then plated on a well in Geltrex-coated 6-well plate when new iPSCs were at passage 1-3. Once iPSC colony morphology became uniformed and stable, mechanical picking up under the microscope was no longer required. The amount of culture media required for culture each day was replaced in 15/50 ml tubes and warmed up at 37° C. in order to keep the original media bottles cold until they are expired. Block heaters for 15/50 ml tubes (VWR) in the culture hood were used. It allowed us to provide cells with warmed media although culture procedures became longer. Also, it helped us minimize risk of contamination compare to using water bath. To change media daily, approximately 2 ml per well (80%) was taken and then freshly prepared media (˜2 ml) was added to keep cells covered by at least 0.5 ml media in 6-well plates, in order to avoid exposure of cells to ambient air to minimize stress. One day before passage, media volume was changed to total 3.5 ml per well in 6-well plate to provide sufficient nutrition to avoid spontaneous differentiation due to high confluency. Regularly, mycoplasma tests were also performed using an automated 96-well luminometer (GloMax 96, Promega) with mycoplasma detection kit (MycoAlert PLUS, Lonza). In addition, osmolality in all media was monitored using Osmette (model 5002, Precision System).

To get iPSC lines adapted to normoxia for tests on cardiac differentiation, semi-confluent culture (30-50%) of feeder-free iPSC lines had been directly transferred from an incubator under hypoxia (5% O₂,) to another incubator with normoxia (20% O₂). However, dead cells were observed due to oxygen stress and it took long to get cells recovered and adapted completely to normoxia. Therefore, the following optimized procedure has been determined as follows: 1) plate feeder-free iPSC lines on CF-1 feeders with E8 media or conventional iPSC media containing KSR; 2) 2-3 days later after plating, transfer the plates from hypoxia to normoxia; 3) pick up uniformed iPSC colonies enzymatically and mechanically and then replace the colonies onto Geltreax-coated plates using E8 media with Y27632 under normoxia. For reproducible results on monolayer cardiac differentiation, an additional 3-5 passages (total 2-3 weeks) has been found to be optimal.

### Transfection

To test lipofection reagents in IMR90 cells, there were no major differences between OptiMEM and DMEM with 10% FBS media (P.S.-free) for TransFectin (BioRad) and Fugene HD (Progema). Procedures using X-fect (Clontech/TaKaRa Bio), Lipofectamine 2000 and Lipofectamine LTX were followed according to manufacturer's instructions. pGW1-YFP (Krey et al., 2013) and pCXLE-EGFP (Addgene #27082) were used to examine transfection efficiency.

iPSC Generation

The three episomal vectors established by the Yamanaka group (Okita et al., 2011) (Addgene. #27077, 27078 and 27080) were used for the reprogramming of control and patient fibroblasts to iPSCs. NHDF (adult normal human dermal fibroblasts, Life Technologies) was used as a control. Briefly, ˜2.5×10⁴ cell/ml were first plated on 4 wells (0.5 ml per well) in non-coated 24-well plate (Corning) using 20% FBS-containing DMEM (high glucose, Glutamax I, with P.S.) under normoxia, and at day two transfection was performed using Lipofectamine LTX (LF-LTX) as follows: the plasmid DNA (0.9 μg per well, ratio=1:1:1, in 50 μl) and 0.9 μl of Plus Reagent was incubated with LF-LTX (2 μl per well, in 50 μl) for 40 min in OptiMEM (Life Technologies), and then the mixture (total ˜103 μl) was transferred to a well containing human fibroblasts immediately after changing DMEM to OptiMEM (0.5 ml per well) in the culture plates. Thirty minutes after lipofection, the media was completely changed to 20% FBS containing DMEM (high glucose, Glutamax I, without P.S.). Further media changes were conducted the next day (day 3) using 20% FBS containing DMEM (high glucose. Glutamax I, with P.S.). On day 5-6, before the fibroblasts became 100% confluent, the cells in a well in 24-well plates were plated on a Geltrex-coated well in a 6-well plate. The next day after cell passage, the culture media was switched to E8 media under hypoxia. Further media changes using E8 were conducted every day while change media even every other until day 14 did not affect the efficiency of iPSC generation (data not shown). Although at day 14-21 some iPSC-like colonies were observed, picking up colonies was done at day 22-27 for most wells of the patient lines. To dissociate colonies, the cells were incubated with dispase (˜1 mg/ml, 1 ml per well) for four min and washed three times using E8 media and then 1.5 ml of E8 media with 10 μM Y27634 was added to the well. To pick up colonies, 27G needle and P200 filtered pipette tip were used under stereomicroscope described as above for regular passage of feeder-free iPSCs. Further mechanical dissociation using pipetting was not required for P0 colonies. Modified protocol is also available with Matrigel (BD Bioscience) and mTeSR1 media (Stem Cell Technologies) for the reprogramming of human skin fibroblasts (data not shown). The same protocol could be used to reprogram hair keratinocytes into iPSCs. In case of reprogramming of human hair follicle keratinocytes (FK, ScienCell, #2440), culture in 20% FBS-containing DMEM (at day 2-6) after lipofection provided iPSC colonies while keratinocyte media (ScienCell, #2101) did not work for reprogramming. The protocol was also used to derive iPSCs from several patient fibroblasts from patients with congenital heart diseases. Patient genetic information is listed in Table 2 (Song, et al. Stem Cells Transl Med. 4(5):468-75 (2015)).

iPSC Characterizations

To examine pluripotent gene expression, we placed the iPSCs on 15-mm round coverslips (Warner Instruments, CS-15R) coated with Geltrex in 24-well plates. The iPSCs were fixed using 4% paraformaldehyde (PFA, Electron Microscopy Sciences, EM grade), 2% sucrose in phosphate buffered saline (PBS) for 20 min at room temperature and washed three times with PBS. The cells were then blocked with a solution containing 3% bovine serum albumin (BSA. Sigma-Aldrich) and 0.1% Triton X-100 (Sigma-Aldrich) in PBS for anti-TRA-1-60 and TRA-1-81 antibody staining for 30 min at room temperature. The samples were then washed three times using 1% BSA in PBS and incubated with the anti-TRA-1-60 and TRA-1-81 antibody (both, 1/200 dilution, Millipore) for at least 24 h at 4° C. After incubation with primary antibodies, the iPSC samples were washed three times, and incubated in secondary antibody. Alexa Fluor 594 (Molecular Probes, 1/1,000 dilution, 2 mg/ml) for 30 min at room temperature, washed with 1% BSA in PBS three times (˜5 min each), stained with Hoechst 33285 (Molecular Probes, 1/10,000 dilution in PBS) and washed with 1% BSA in PBS six time and with PBS once and then mounted on slides using Poly/Mount (Thermo Scientific). Images were acquired using a 20× objective lens (CFI Plan Apochromat Lambda DM. NA 0.75, Nikon) on the stage of a Nikon Eclipse TiU inverted microscope operated by MetaMorph (Molecular Devices).

Karyotyping of iPSCs was archived in WiCell (Wisconsin). The iPSCs were plated on CF-1 feeder using conventional iPSC media containing KSR in T25 flasks and then shipped out since we found that many feeder-free iPSCs that have been maintained under hypoxia were dying during shipping due to oxygen stress. For RT-PCR using primer sets (Okita et al., 2011; Takahashi et al., 2007; Yazawa et al., 2011), RNA from fibroblasts and iPSCs was prepared using the RNeasy Mini kit and RNase-Free DNase set (Qiagen). cDNA was synthesized from 1 μg RNA using the SuperScript III First-Strand Synthesis System for RT-PCR (Life Technologies). The cDNA (21 μl) was diluted with DNase-free water (Invitrogen) at 1:5 and 1 μl of the samples was used for conventional RT-PCR with Ex taq (Clontech/TaKaRa Bio). For quantitative RT-PCR, SYBR Advantage qPCR Premix (Clontech/TaKaRa Bio) and StepOnePlus real time PCR systems (Life Technologies) were used.

TABLE 1

beating cells

BIO 20% O₂

| | | | |
|---|---|---|---|
| GSK3 inhibitor IX | 1 | μM | Yes |
| Calbiochem | 2 | μM | Yes |
| | | | only around the edge of well |
| Cat # 361550 | 4 | μM | No |
| Lot # D00148116 | 8 | μM | No |

CHIR 20% O₂

| | | | |
|---|---|---|---|
| CHIR99021 | 5 | μM | Yes |
| | | | only around the center of well |
| Axon MedChem | 10 | μM | No |
| Cat # 1386 | 15 | μM | No |
| Lot # 6 | 20 | μM | No |

5 μM CHIR & 1 μM BIO

| 5% O₂ | | | Yes |
|---|---|---|---|
| | | | few small regions |
| | 5% O₂ | 20% O₂ | Yes |
| | (day 1-4) | (day 5-) | very few small regions |

| 20% O₂ | Yes |
|---|---|
| | all regions |

Teratoma formation assays were performed by injecting subcutaneously confluent iPSCs (in a 10 cm dish) in 20% E8 media, 30% FBS, 50% Geltrex and 10 uM Y-27632 (Calbiochem or TOCRIS) into 8-week-old severe combined immunodeficient (SCID) beige male mice (Charles River Laboratories). The SCID-beige mice were sacrificed at 6-8 weeks after injection, the tumors were weighted, dissected, fixed with PBS containing 4% PFA and 10-30% sucrose and embedded in Tissue-Tek OCT compound (Sakura Finetek). Sections were stained with Hematoxylin and Eosin-Y (Thermo Scientific). All animal protocols and handling for the teratoma formation assay were performed following the guidelines established by Columbia Institutional Animal Care and Use Committee under our approved protocol (for Y.Y and M.Y, #AC-AAAF5655). To examine differentiation potentials of generated iPSC lines used for further experiments, neural differentiation and cardiac differentiation were conducted as reported previously, using human recombinant proteins, EGF, BDNF and NT-3 (all from Peprotech) (Pasca et al., Nature medicine 17, 1657-1662. (2011)) and Wnt3A (#5036-WN-010/CF, R & D Systems) (Yazawa et al. Nature 471:230-234 (2011)).

### Monolayer Cardiac Differentiation and Characterization

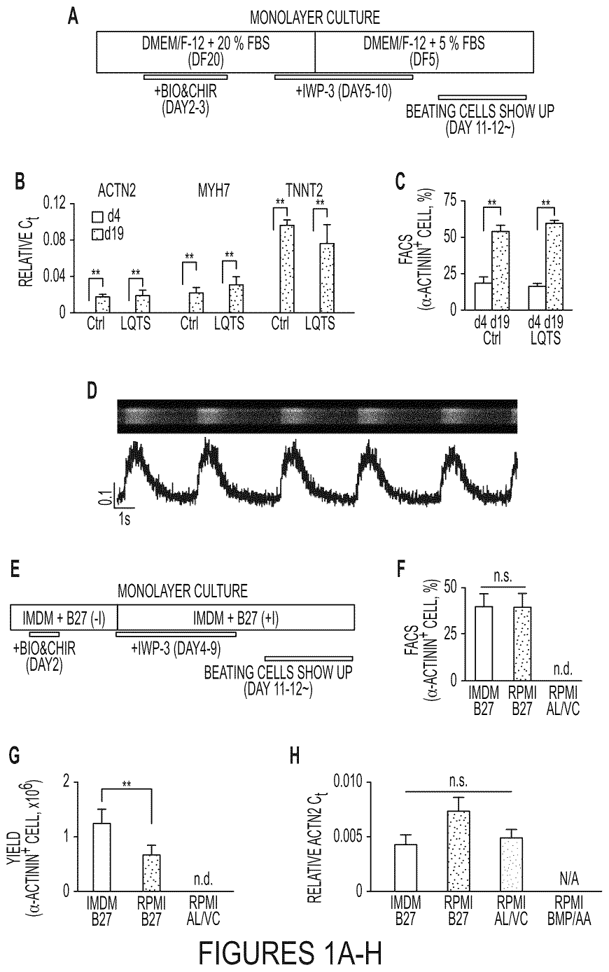

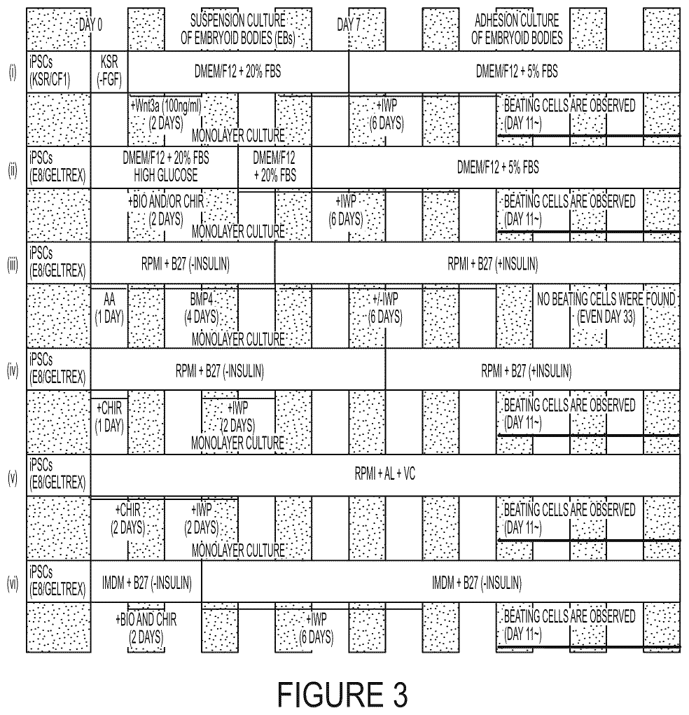

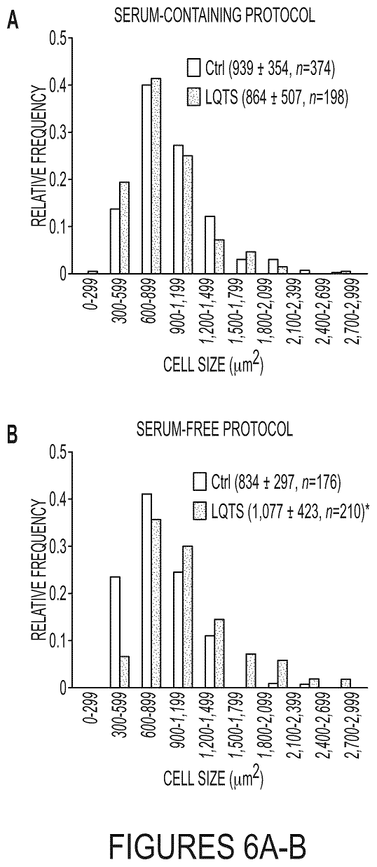

CHIR99021 (GSK3 inhibitor, #1386, Lot #6 & #7, Axon MedChem) and BIO (GSK3 inhibitor IX, #361550, Lot #D00148116, Calbiochem) (Minami et al., Cell reports 2, 1448-1460(2012)), IWP-3 (Wnt inhibitor, #SML0533, Lot #102m4613V, Sigma-Aldrich) resolved in Dimethyl Sulfoxide (DMSO, #276855, Sigma-Aldrich) were used for monolayer differentiation of iPSCs plated on Geltrex in 6-well plate or 10 cm dish. To prepare differentiating culture media, only one freeze and thaw cycle was confirmed for all aliquots of small compounds CHIR99021 and BIO and recombinant human growth factors. Twice freeze and thaw cycle was confirmed for IWP-3 aliquots. For the differentiation protocol with serum-containing media, DF20/5 media was used for cardiac differentiation and culture: DMEM/F-12 (with GlutaMax I) containing 20% or 5% Hyclone FBS, 1% non-essential amino acid, P.S. and 0.1 mM bME. Additional D-glucose was added to only DF20 as high glucose media (final 4.5 g/l) to enhance mesoderm differentiation from Day 1 to Day 4. The full optimized differentiation protocol is shown in FIG. 1A and FIG. 3(ii). The optimization of culture conditions (dosing of CHIR and BIO and oxygen level) is summarized in Table 1. For BIO and CHIR treatment, media was changed everyday. For IWP-3 treatment, media was changed every other day. DF5 media was used for the maintenance of cardiomyocytes after differentiation (Day 11˜) and media was changed every other day. Changed media using DF20 with small compounds required ˜3 ml per well in 6-well and ˜15 ml per 10 cm dish every other day since adding 2-2.5 ml of fresh media to a well of 6-well plates was not sufficient for high confluence of cells during differentiation into cardiomyocytes, resulting in lower efficiency of cardiac differentiation. Several lots of USDA-approved FBS (from Thermo Hyclone and Gibco) were tested after we found that CHIR/BIO in DEME/F-12 worked with FBS, and HyClone FBS (not heat-inactivated, #SH30071.03, Lot #AYE161479, Thermo Scientific) was only used for further monolayer differentiation since we found that this lot of Hyclone FBS provided slightly higher efficiency to generate cardiomyocytes reproducibly than other FBS. For the differentiation protocol without serum containing media, Iscove's Modified Dulbecco Media (IMDM, Sigma-Aldrich) with B27 supplement (-minus insulin version, Life Technologies) was used for mesoderm differentiation (Day 1 to Day 3) and IMDM media with B27 (regular version, Life Technologies) was used for further differentiation and maintenance (Day 4˜). The full protocol was showed in FIGS. 1E and 3(vi). For the comparison between different serum free monolayer cardiac differentiation protocols (FIG. 3), RPMI1640 media (Sigma-Aldrich) and Activin A and BMP4 (R & D Systems), B27 supplement (both regular & minus insulin version, Life Technologies), human albumin (Sigma) and L-ascorbic acid 2-phosphate (Sigma-Aldrich) were used to make the corresponding differentiation media. In case of using DMEM/F-12 media for serum-free protocol, all cells died during early stage of cardiac differentiation (data not shown). All images and movies were taken in the tissue culture room using TS100F-LED microscope operated by Nikon NIS-Elements with objectives (4×, CFI Plan Fluor DL, NA 0.13; 10×, CFI Achromat ADL, NA0.25; 20×, CFI Achromat LWD ADL, NA 0.4, Nikon). Antibodies to sarcomeric alpha-actinin (clone #EA-53, Sigma-Aldrich) were used for immunocytochemistry of human cardiomyocytes (3-4 weeks later after cardiac differentiation). The cell size was analyzed blindly by E. Uche-Anya using Fiji software (FIG. 6). FACS analyses were performed in Columbia Center for Translational Immunology (CCTI) using a BD FACS Calibur using control and patient cardiomyocytes at day 19. Standard protocol for FACS was used first with 0.5% saponin (Sigma-Aldrich) for permeabilization at blocking step and next with sarcomeric alpha-actinin antibody (Sigma-Aldrich, clone #EA-53, 1/200 dilution) or cardiac troponin T (clone #13-11, Thermo Scientific, 1/500 dilution) and goat anti-mouse Alexa Fluor 488 secondary antibody (Life Technologies, 1/500) in FACS buffer (0.1% saponin, 2% FBS and 2 mM EDTA in Hanks buffer in HEPES, HBSS). The FACS data analyses were blindly performed by J. Feng. The identical cell samples were also used before fixation, for RNA/cDNA preparation and gene expression profiling using qPCR.

### Confocal Microscopy Calcium Imaging:

Monolayer or aggregates of beating cardiomyocytes were dissociated into single cells or small clusters and placed in 35 mm glass bottom culture dishes (7 mm diameter No. 1.5 glass, MatTek, #P35G-1.5-7-C) coated with gelatin (Sigma-Aldrich) two days before Ca²⁺ imaging. To check dissociation quality for especially LQTS cardiomyocytes, a key factor was whether some large aggregates showed spontaneous contraction the next day after dissociation (therefore, we did not completely dissociate all monolayers of cardiac cells into single cells to minimize damage of single-conditioned cells) because the LQTS phenotype was so severe due to calcium overload that many patient cells might just die because of mechanical stress in addition to the cellular phenotypes. On the day of confocal imaging, cardiomyocytes were loaded with FluoForte dye in HBSS with dye efflux inhibitor for 1 hour at room temperature according to the manufacturer's instruction manual (FluoForte™ Calcium Assay Kit, Enzo Life Sciences). FluoForte calcium dyes provided higher signal and lower toxicity in iPSC-derived cardiomyocytes compared to Fluo-4 AM, which was previously used for iPSC disease modeling of LQTS (Yazawa et al., 2011) (data not shown). Next, glass-bottom dishes were washed three times with normal Tyrode solution containing 1.8 mM CaCl₂) and 10% FBS. Ca²⁺ imaging was conducted with a Nikon A1 scanning confocal microscope on an Eclipse Ti microscope stand with 100× lens (NA 1.45) operated by NIS element software (all from Nikon). The excitation spectrum was 488 nanometer and the standard FITC filter was used for emission. The confocal pinhole was set at 1 Airy unit. The line was placed at the center of each cell and line scans were acquired at a sampling rate of 1.9 ms per line for 20 s recordings at room temperature. For drug candidate testing for LQTS patient-derived cardiomyocytes, stock solutions of R-roscovitine (Sigma Aldrich) were prepared in DMSO. Since at room temperature, frequent slower or halting contractions were observed in control and patient cardiomyocytes, 10% serum was added to the recoding solution to observe spontaneous beating in the cells. Therefore, Tyrode solution containing 10% FBS, 1.8 mM CaCl₂), and 0.2% DMSO was used as control solution and washing buffer. Working solution for roscovitine was prepared with Tyrode solution containing 1.8 mM CaCl₂) and 10% FBS. The final concentration of DMSO in working solution was 0.2%. Line scans were acquired two times (before drug treatment and 20 min after drug treatment) from the same cell using the same position with stage controller in the confocal microscope. All scanning settings were held constant throughout the experiment. Fiji software, Excel (Microsoft) and programs developed were used to analyze irregularity of spontaneous Ca²⁺ transients (Yazawa et al. Nature 471:230-234 (2011)).

### R-GECO1 and ArcLight Preparation and Imaging

Plasmids of R-GECO1 (Addgene #32462) (Zhao et al., 2011) and ArcLight (Addgene #36857) (Jin et al., 2012) were used as template to amplify the cDNA using Phusion polymerase (Thermo Scientific) with primer sets that allowed us to add restriction enzyme site EcoRI and Kozak sequence before the start codon (without nuclear localization signal for R-GECO) and another site XhoI after the stop codon. These PCR products were subcloned into lentiviral vector that was prepared from LV-SD-Cre (Addgene, #12105, no longer available currently) digested with EcoRI and XhoI. XL-10 Gold competent cells (Agilent) transformed with these LV-SD vectors were inoculated at 24-30° C. The purified LV-SD vectors were transfected together with pCMV-dR8.2 dvpr and pCMV-VSV-G (Addgene #8455 & 8454) as follow: HEK293T cells that were plated at 5×10⁵ cell/ml (2 ml per well) one day before were transfected with total 3.4 μg per well (ratio, LV-SD:dR8.2:VSV-G=8:8:1) and LF-2K (8.0 μl per well) in a 6-well plate. The next day the media was changed. Two days after transfection, culture media was harvested (again fresh media was added to transfected HEK 293T cells) and filtered using 0.45 μm PES filter (VWR) and then directly added to wells containing monolayer cardiomyocytes (at 2-3 weeks after inducing cardiac differentiation). No polybrene was used for viral infection. Seventy-two hours after transfection, the culture media was again collected and used as secondary infection. The next day media was changed to DF5 media for infected cardiomyocytes. In case of serum-free condition, Lenti-X concentrator (TaKaRa/Clontech) was used for media change, following the manufacture's instruction. Three to five days after the secondary infection, monolayer cardiomyocytes were transferred to 35 mm glass bottom culture dishes (7 mm diameter, No. 1.5 glass) coated with Geltrex. Nikon Ti-U epi-fluorescent microscope connected to EMCDD Digital Monochrome camera (Evolve, EVO-512-M-FW-16-AC, 512×512, Photometrics) was used with LED light source (Lumencore Spectra X 6 line LED system, with Chroma excitation mounted filters, ET395/25x, ET470/30, ET550/15x, ET640/30x;) with dichroic mirror and emission filter (dichroic mirror, D/F/Cy3/Cy5pc; emission, ET430/36 513/44 595/41 719/105) operated by MetaFlour (Molecular Devices, acquisition: exposure time, 20 ms; gain 3 (6×); transfer speed, 5 MHz, Image size: 512×512; binning, 1.0). Temperature of the bath solution was maintained at approximately 35° C. using a digital temperature controller (TC-344B, Warner Instruments) for the duration of the experiment. Nikon objective lens 40× (CF1 S Fluor, NA 0.90) for cell clusters and 60× (CFI Plan Apo Lambda H, NA 1.40) for single cells were used to capture two fluorescent signals as R-GECO1 (illumination control. 10 in 0-255, cy3) and ArcLight (150 in 0-255, FITC, 4 or 8 frames per sec). Isoproterenol (10 μM, Sigma Aldrich) and R-roscovitine (0.5 and 5 μM) were used for fluorescent imaging. The effect of serum concentration on the imaging using ArcLight and R-GECO1 in control and patient cells was not observed at approximately 35° C. Upstroke velocity was analyzed based on relative ArcLight fluorescent signal, demonstrating dFᵣ/dtₘₐₓ, Instead of voltage (V), Fᵣ=100*(F−F₀)/(Fₚₑₐₖ−F₀) was used (Fᵣ range, 1-100). Daily imaging in cardiomyocytes infected at day 15 was conducted for 18 days (day 23-40). Additional recordings were performed at day 90 to examine low long ArcLight and R-GECO1 signals are available.

### Electrophysiological Recording

Whole-cell patch-clamp recordings of human cardiomyocytes generated from iPSCs were conducted using MultiClamp 7000B (Molecular Devices) and an inverted microscope (Ti-U, Nikon) used for ArcLight and R-GECO1 imaging. The glass pipettes were prepared using borosilicate glass (Sutter Instrument, BF150-110-10) using a micropipette puller (Sutter Instrument. Model P-97). Simultaneous current-clamp recording with optical imaging were conducted using pClamp 10 and MetaFluor softwares (Molecular Devices) in normal Tyrode solution containing 140 mM NaCl, 5.4 mM KCl, 1 mM MgCl₂, 10 mM glucose, 1.8 mM CaCl₂) and 10 mM HEPES (pH 7.4 with NaOH at 25° C.) using the pipette solution: 120 mM K D-gluconate, 25 mM KCl, 4 mM MgATP, 2 mM NaGTP, 4 mM Na₂-phospho-creatin, 10 mM EGTA, 1 mM CaCl₂) and 10 mM HEPES (pH 7.4 with KCl at 25° C.).

### Reprogramming

Plating density of fibroblasts (˜1.25×104 cells per well in 24-well plate) is important to get reproducible results in case of fast-proliferating patient fibroblasts (our case, growth in CDH05-0011 was much faster than the others). This is because changing to E8 media should be ideally done five to six days after lipofection. If changing the media from DMEM to E8 media is done too early after lipofection, the number of iPSC colonies might decrease. Not only E8 media but also mTeSR1 media (Stem Cell Technologies) were tested and confirmed to generate iPSC even under normoxia as well as hypoxia while conventional iPSC culture media containing KSR worked only under hypoxia. To skip adaptation phase of iPSCs from hypoxia and normoxia, reprogramming under normoxia might be helpful though there is a well-known reduction in reprogramming efficiency under normoxia compared to hypoxia (Yoshida et al., 2009). For future automated approach for cellular reprogramming, we used a 96-well plate as well as 24-well plate and generated a new iPSC line (CH5-E2-1 line).

### Preparation of Small Compounds

Dimethyl Sulfoxide (DMSO) quality is critical to get reproducible results using small compounds, especially CHIR99021 (CHIR, #1386, lot #6 & #7, Axon MedChem). DMSO should be kept from ambient air. Needle (18G) and syringe were used to withdraw DMSO (#276855, Sigma-Aldrich) to minimize exposure to the air rather than opening the bottle. For instance, to dissolve CHIR (5 mg) in ˜1.075 ml of DMSO completely as 10 mM stock requires warming in a 37° C. water bath. Although only vials from two lots (#6 & #7) of this compound were used for this study, we found that second and later preparations of CHIR stock aliquots (˜50 tubes, 20 μl per tube) were slightly better than the first ones since warming to dissolve it completely was carefully monitored and incubation time was minimized. Therefore, since we found that protocol using 3 μM CHIR with 1 μM BIO (day 2-4) was also available to differentiate iPSC into cardiomyocytes, a pilot test using both 3 μM and 5 μM CHIR is highly recommended together with 1 μM BIO at day 2-3 for the serum-containing protocol or at day 2 for the serum-free protocol. All aliquots of CHIR and BIO (10 mM stock in DMSO) were stored at −80° C. until used. Compared to CHIR and BIO, IWP-3 (2 mM stock in DMSO) is relatively easy and stable to use and store. Aliquots of IWP-3 stock (originally stored at −80° C.) were in fact returned to the −20° C. freezer a maximum of two times during six-day treatment (day 5-10) for cardiac differentiation.

The present invention also provides a kit for promoting cardiac differentiation comprising one or more GSK3 signaling inhibitors and/or one or more WNT signaling inhibitors which is used for the method of inducing cardiac differentiation of the invention.

Additionally, a kit can comprise one or more GSK3 signaling inhibitors and/or one or more WNT signaling inhibitors, along with cardiac differentiated stem cells prepared according to the methods described herein.

### Example 1

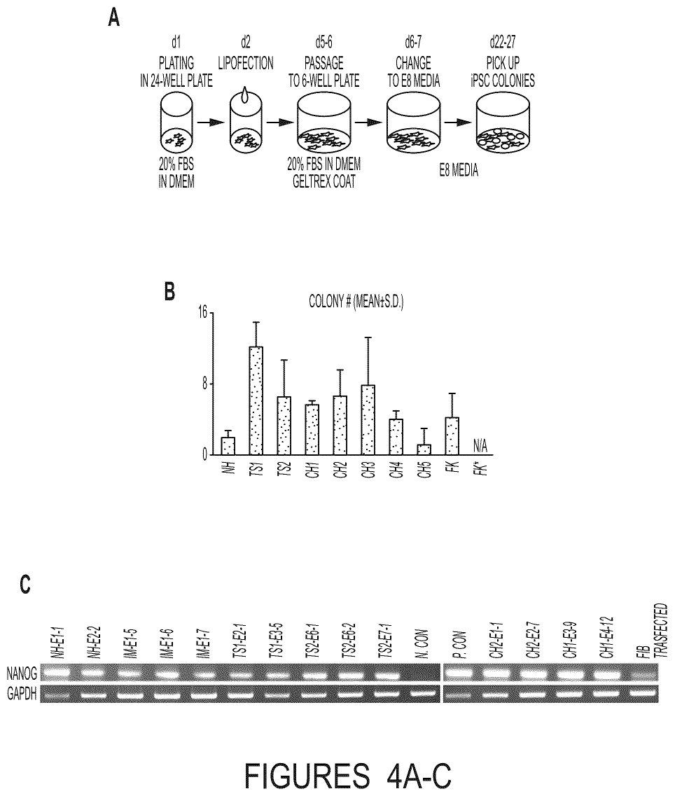

Transfection using electroporation is widely used for the reprogramming of human fibroblasts with the episomal vectors to generate integration/virus-free iPSCs (Okita et al., 2011; Yu et al., 2009). However, electroporation requires equipment, expertise and millions of cells to minimize toxicity and optimize the efficiency of gene transduction in each patient sample. In contrast, lipofection is a relatively simple procedure compared to electroporation, although repeated lipofection of modified RNA is required for reprogramming (Warren et al., Cell stem cell 7, 618-630(2010)). To develop a simple and inexpensive protocol for the reprogramming to pluripotency, we first optimized transfection in IMR90 human embryonic lung fibroblasts in 24-well plates using Lipofectamine 2000 (LF-2K). LF-2K has been used for gene transduction in mammalian primary cells such as cortical neurons (Krey et al., Nature neuroscience 16, 201-209, (2013)) to express yellow fluorescent protein (YFP). We found that exposing IMR90 cells to plasmid-LF-2K complexes in OptiMEM for −30 min using 20% fetal bovine serum (FBS) in media for recovery provided higher expression of YFP and lower cell death caused by toxicity of lipofection. To determine whether single lipofection using LF-2K is sufficient for reprogramming of IMR90 to induced Pluripotent Stem Cells (iPSCs), we next tested this method using the established episomal vectors (Okita et al., Nature methods 8, 409-412 (2011)), and we found that iPSC lines could be derived using a 24-well plate under hypoxia (Yoshida et al., Science 324, 797-801 (2009)) using Essential 8 (E8) media (Chen et al., Nature methods 8, 424-429 (2011)). We further optimized conditions and reagents to use lipofection in patient fibroblasts because we found that one out of four wells in 24-well plate failed to generate iPSCs and because IMR90 embryonic fibroblasts are known to be easier to reprogram than human adult fibroblasts (Yazawa et al. Nature 471:230-234 (2011)). After testing several lipofection reagents to transfect an episomal vector, we found that human fibroblasts transfected using Lipofectamine LTX (LF-LTX) showed higher gene transduction and faster recovery compared to those transfected with other lipofection reagents. Therefore, we used LF-LTX to transfect the episomal vectors to control and patient fibroblasts in a 24-well plate, and we generated multi-independent lines of control and patient-specific iPSCs from patients with long QT syndrome (LQTS, type 8, Timothy syndrome) (Yazawa et al. Nature 471:230-234 (2011)), and congenital heart diseases (CHD) (Table 2). The full protocol is shown in FIG. 4A and the efficiency is shown in FIG. 4B.

TABLE 2
Long QT Syndrome (type 8, Timothy syndrome)

| Patient code | Fibroblast ID | gene | mutation | gender | phenotpes |
|---|---|---|---|---|---|
| TS1 | TS7643 | CACNA1C | G406R | female | LOTS (ref. Yazawa et al., 2011) |
| TS2 | TS9862 | CACNA1C | G406R | male | LQTS (ref. Yazawa et al., 2011) |

Congenital heart disease (CHD) patients

| Patient code | Fibroblast ID | gene | mutation* | gender | phenotpes |
|---|---|---|---|---|---|
| CH1 | CDH03-0005 | GPC1 glypican 1 | G418R | female | CDH**, Ebstein's Deformity of tricuspid valve |
| CH2 | CDH01-0634 | PRKACB | R106X | male | CDH**, Ventricular septal defect |
| | | protein kinase, | | | |
| | | cAMP-dependent, | | | |
| | | catalytic, beta | | | |
| CH3 | CDH03-0001 | PPAPDC2 | V275E | male | CDH**, Atrial septal defect, right limb hypoplasia, |
| | | phosphatidic acid | | | absence right scapula |
| | | phosphatase type 2 | | | syndactyly fingures 3 & 4 of right hand |
| | | domain containing 2 | | | |
| CH4 | CDH01-0158 | ROBO1 | R310X | female | CDH**, ventricular septal defect |
| CH5 | CDH05-0011 | INHBB | G418R | male | CDH**, congenital hydrocephalus, |
| | | | | | seizures, chronic lung disease |

*All identifided mutations are de novo.
**Congenital diaphragmatic hernia (CDH) is also found in the patients as well as congenital heart diseases.

We characterized these lines and confirmed that the iPSCs showed pluripotency, using immunocytochemistry, karyotyping, teratoma formation assay, gene expression profile (FIG. 4C) and in vitro differentiation.

To examine if the method is generally applicable to other cell types, especially to less invasive cell sources, we used LF-LTX to transfect the vectors to human hair follicle keratinocytes on the same scale. We found that we could generate iPSCs from the keratinocytes using the same recovery media used for fibroblasts after the lipofection while no iPSCs were generated when keratinocyte-specific media was used on day 2-6 after lipofection (FIG. 4B). These results demonstrate that the optimized protocol for reprogramming is efficient, reproducible and applicable for the generation of iPSCs so as to derive a variety of disease models using patient skin fibroblasts and hair keratinocytes. The iPSCs derived from the skin fibroblasts of Timothy syndrome patients using the optimized protocol are the materials for the cardiac differentiation protocols as used in embodiments of the present invention.

The present invention relates in part to the new monolayer protocols for the cardiac differentiation of human iPSCs. In certain embodiments, these monolayer cardiac differentiation protocols relate to differentiating human iPSCs derived from healthy control fibroblasts or from patient cells, and in particular exemplified by differentiating Timothy syndrome patient specific iPSCs into cardiomyocytes under either serum-free or serum containing conditions. One protocol involves the use of Fetal Bovine Serum (FBS) and is referred to as Serum-containing protocol in Example 2. A second protocol does not involve the use of serum and is referred to as Serum-free protocol in Example 3.

### Example 2

### Serum Containing Protocol

The outline of the serum-containing protocol is shown in FIG. 1A. The iPSC lines are cultured in a 37° C., 20% oxygen incubator with commercial Essential 8 media in 6 well plates or 100 cm dishes coated with Geltrex before differentiation. The protocol involves the use of three chemicals, CHIR99021 (CHIR, GSK3 inhibitor, Axon MedChem) and BIO (GSK3 inhibitor IX, Calbiochem) and IWP-3 (Wnt inhibitor, Sigma-Aldrich). A single use of four different doses of BIO (1, 2, 4, 8 μM) and Four different doses of CHIR (5, 10, 15, 20 μM) for differentiation were tested during the optimization of this protocol (Table 1). The result demonstrated that single use of 1 μM BIO and 5 μM CHIR at day 2 and day 3 during the differentiation provided beating cardiomyocytes. We found that a combination of BIO and CHIR at day 2 and day 3 during differentiation further enhanced the differentiation efficiency and the dose of CHIR could be further reduced when it was used together with BIO. For the optimized protocol, the dose of BIO is 1 μM. The default dose of CHIR for this protocol is 3 μM, while titration of the dose of CHIR from 1 μM to 5 μM is recommended for optimizing this protocol for different human iPSC lines. The dose of IWP-3 is 2 μM. All chemicals are prepared in Dimethyl Sulfoxide (DMSO). We also tested 5% and 20% oxygen conditions or a combination of both to further optimize the protocol and found that 20% oxygen condition was more optimal for the cardiac differentiation (Table 1). DF20/5 media was used for cardiac differentiation and culture. The components of DF20/5 media are DMEM/F-12 (with GlutaMax 1) containing 20% or 5% Hyclone FBS, 1% non-essential amino acid, P.S. and 0.1 mM bME. Additional D-glucose was added to only DF20 as high glucose media (final 4.5 g/1) to enhance mesoderm differentiation from Day 1 to Day 4. BIO and CHIR treatment was administered at day 2 to day 3. The media containing BIO and CHIR was changed every day. For 2 μM IWP-3 treatment at day 5 to day 11, media was changed every other day. At day 5-7, DF20 media was used with IWP-3. DF5 media was used at day 7˜ for IWP-3 treatment and maintenance, and the media was changed every other day. Beating cardiomyocytes in monolayers are observed from Day 11 (FIG. 1A). Changed media with small compounds required ˜3 ml per well in 6-well and ˜15 ml per 10 cm dish every other day since adding 2-2.5 ml of fresh media to a well of 6-well plates was not sufficient for high confluence of cells during differentiation into cardiomyocytes, resulting in lower efficiency of cardiac differentiation. Several lots of USDA-approved FBS (from Thermo Hyclone and Gibco) were tested after finding that CHIR/BIO in DEME/F-12 worked with FBS, and HyClone FBS was only used for further monolayer differentiation since we found that this lot of Hyclone FBS provided slightly higher efficiency to generate cardiomyocytes reproducibly than other FBS. The cardiomyocytes derived from both control and Timothy syndrome patient iPSCs showed organized sarcomere arrangements and high expressions of cardiac markers (FIG. 1B). Fluorescence activated cell sorting (FACS) analysis suggest that the differentiation efficiency of this protocol is around 50%-60% (FIG. 1C).

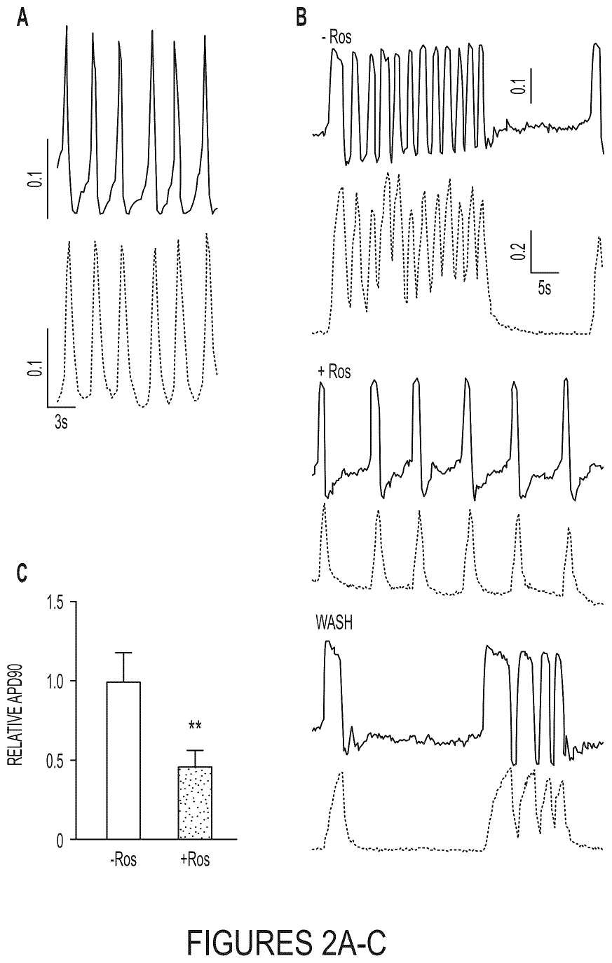

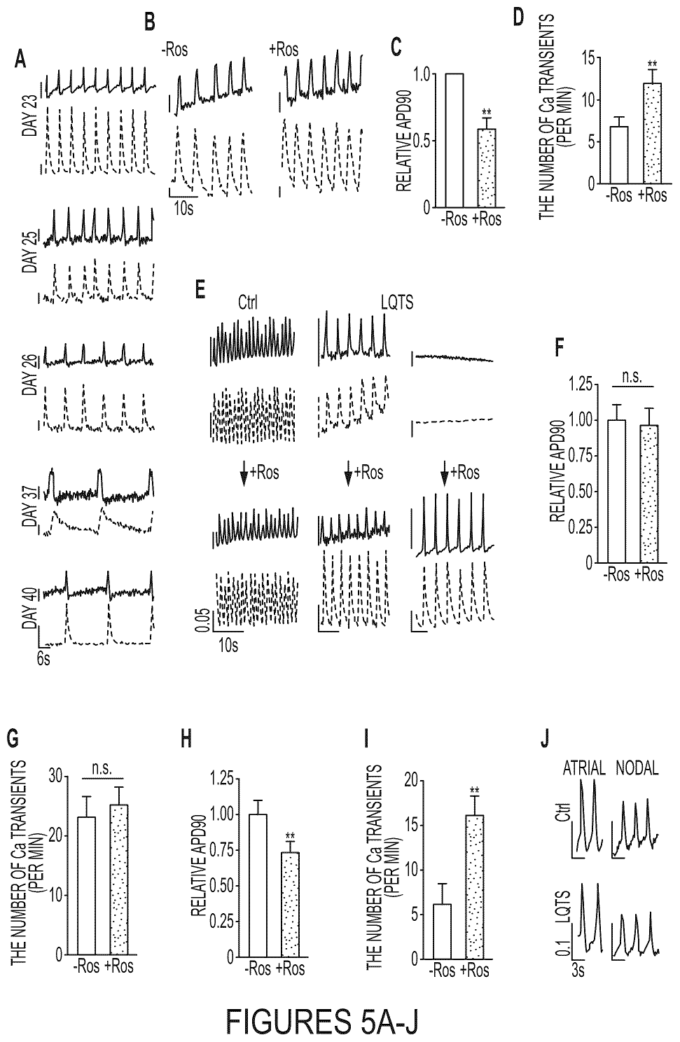

A dual optical imaging system using two genetically encoded indicators, ArcLight for voltage and R-GECO1 for calcium transients, has been developed to efficiently phenotype the patient specific iPSC-derived cardiomyocytes derived from the serum containing protocol (Song, et al. Stem Cells Transl Med. 4(5):468-75 (2015)). The recordings using the dual optical imaging system showed that the cardiomyocytes derived from control iPSCs showed regular action potentials and calcium handlings (FIG. 1D and FIG. 2A). In contrast, the cardiomyocytes derived from Timothy syndrome patient iPSCs exhibited prolonged action potentials and abnormal calcium handling (FIG. 2B and FIG. 5). Those phenotypes of Timothy syndrome patient iPSCs derived cardiomyocytes can be rescued by 5 μM Roscovitine treatment (FIGS. 2B-2C and FIG. 5). These data demonstrate that this culture protocol is applicable for differentiating both control and patient specific human iPSCs into cardiomyocytes, and the phenotypes of the cardiomyocytes are conserved after differentiation.

Advantages of this culture protocol include: (1) In contrast to other conventional Embryonic body (EB) based protocols, the present protocol is based on feeder-free monolayer iPSC culture. Changing media for monolayer culture and other experimental procedures in this protocol are convenient and reproducible, making it suitable for large-scale production of cardiomyocytes for drug screens. (2) Previous protocols for cardiac differentiation have all involved the use of recombination proteins such as activin A, Bone morphogenetic protein (BMP) 4 and other recombinant proteins. The cost and instability of the recombinant factors make those protocols not ideal for large scale production of cardiomyocytes for drug screening purposes. In contrast, the present protocols utilize inexpensive, defined chemicals at an unexpectedly low dose, making them more suitable for large scale production of human cardiomyocytes. It is noted that previous attempts to utilize chemicals for the cardiac differentiation of human iPSC showed that they could only be applied to control human iPSCs with high efficiency but not patient human iPSC's. The present optimized protocols as shown herein are applicable for both control and patient hiPSCs with relatively high differentiation efficiency. This allows for the use of the serum containing protocol to derive control cardiomyocytes for cardiac toxicity tests and the patient cardiomyocytes for drug screens to identify new therapeutics.

### Example 3

### Serum-Free Protocol

The outline of this protocol is shown in FIG. 1E. The iPSC lines are cultured in a 37° C., 20% oxygen incubator with commercial Essential 8 media in 6 well plates or 100 cm dishes coated with Geltrex before differentiation. Three chemicals, CHIR99021 (CHIR, GSK3 inhibitor, Axon MedChem) and BIO (GSK3 inhibitor IX, Calbiochem) and IWP-3 (Wnt inhibitor, Sigma-Aldrich) are utilized in the serum-free protocol. The dose of each compound is the same as that used in the serum-containing protocol. BIO and CHIR are used for inducing mesoderm differentiation at Day 2. IWP-3 is used from Day 4 to Day 9. All chemicals are prepared in Dimethyl Sulfoxide (DMSO). IMDM-B27 (with or without insulin) media was used for cardiac differentiation and culture: B27 supplement with or without insulin and P.S. were added to Iscove's Modified Dulbecco's Medium (IMDM) media. IMDM B27 (without insulin) is used from Day 1 to Day 3 and IMDM B27 (with insulin) is used from Day 4 to Day 9 and cell maintenance. Beating cardiomyocytes are observed from Day 10. FACS analysis suggest that the differentiation efficiency of this protocol is moderate (˜40%) (FIG. 1F). We compared our serum-free protocol with three other currently available serum-free differentiation protocols (Lian, et al. Nature protocols 8, 162-175 (2013); Laflamme, et al. Nature biotechnology 25, 1015-1024(2007); Burridge, et al. Nature methods 11, 855-860(2014))(FIG. 1 and FIG. 3). The RPMI-BMP/AA protocol with recombinant factors didn't provide us with any cardiomyocytes. Further, the efficiency of the present methods appears to be comparable with the widely used RPMI-B27 protocol (Lian, et al. Nature protocols 8, 162-175 (2013)), and the absolute yield of cardiomyocytes from the present serum-free protocol is higher than the RPMI-B27 protocol (as indicated in FIGS. 1F-1H). Additionally, the presently described protocol is applicable for both control and patient human iPSCs. The dual optical imaging system was used to examine the phenotypes of the Timothy syndrome cardiomyocytes derived using the serum-free protocol. The phenotypes observed when using the optimized serum-free protocol is consistent with phenotypes from our previous report (Yazawa et al. Nature 471:230-234 (2011)). 5 μM Roscovitine treatment rescued the phenotypes of Timothy syndrome cardiomyocytes derived using the serum-free protocol (FIGS. 5E, 5H&5I). The serum free protocol allows us to generate human cardiomyocytes in a defined environment. The cardiomyocytes generated with the serum free protocol can also be used therapeutically for cardiac cell regeneration and for unveiling additional phenotypes of the cardiomyocytes such as the hypertrophy phenotype in Timothy syndrome cardiomyocytes (FIGS. 6A&6B).

### REFERENCES

Bellin, M., Casini, S., Davis, R. P., D'Aniello, C., Haas, J., Ward-van Oostwaard, D., Tertoolen, L. G., Jung, C. B., Elliott, D. A., Welling, A., et al. (2013). Isogenic human pluripotent stem cell pairs reveal the role of a KCNH2 mutation in long-QT syndrome. The EMBO journal 32, 3161-3175.
Burridge, P. W., Matsa, E., Shukla, P., Lin, Z. C., Churko, J. M., Ebert, A. D., Lan, F., Diecke, S., Huber, B., Mordwinkin, N. M., et al. (2014). Chemically defined generation of human cardiomyocytes. Nature methods 11, 855-860.
Chen, G., Gulbranson, D. R., Hou, Z., Bolin, J. M., Ruotti, V., Probasco, M. D., Smuga-Otto, K., Howden, S. E., Diol, N. R., Propson, N. E., et al. (2011). Chemically defined conditions for human iPSC derivation and culture. Nature methods 8, 424-429.
Chong, J. J., Yang, X., Don, C. W., Minami, E., Liu, Y. W., Weyers, J. J., Mahoney, W. M., Van Biber, B., Palpant, N. J., Gantz, J. A., et al. (2014). Human embryonic-stem-cell-derived cardiomyocytes regenerate non-human primate hearts. Nature.
Dambrot, C., Braam, S. R., Tertoolen, L. G., Birket, M., Atsma, D. E., and Mummery, C. L. (2014). Serum supplemented culture medium masks hypertrophic phenotypes in human pluripotent stem cell derived cardiomyocytes. Journal of cellular and molecular medicine 18, 1509-1518.
Itzhaki, I., Maizels, L., Huber, I., Zwi-Dantsis, L., Caspi, O., Winterstem, A., Feldman, O., Gepstein, A., Arbel, G., Hammerman, H., et al. (2011a). Modelling the long QT syndrome with induced pluripotent stem cells. Nature 471, 225-229.
Itzhaki, I., Rapoport, S., Huber, I., Mizrahi, I., Zwi-Dantsis, L., Arbel, G., Schiller, J., and Gepstein, L. (2011b). Calcium handling in human induced pluripotent stem cell derived cardiomyocytes. PloS one 6, e18037.
Jin, L., Han, Z., Platisa, J., Wooltorton, J. R., Cohen, L. B., and Pieribone, V. A. (2012). Single action potentials and subthreshold electrical events imaged in neurons with a fluorescent protein voltage probe. Neuron 75, 779-785.
Kattman, S. J., Witty, A. D., Gagliardi, M., Dubois, N. C., Niapour, M., Hotta, A., Ellis, J., and Keller, G. (2011). Stage-specific optimization of activin/nodal and BMP signaling promotes cardiac differentiation of mouse and human pluripotent stem cell lines. Cell stem cell 8, 228-240.
Koyanagi-Aoi, M., Ohnuki, M., Takahashi, K., Okita, K., Noma, H., Sawamura, Y., Teramoto, I., Narita, M., Sato, Y., Ichisaka, T., et al. (2013). Differentiation-defective phenotypes revealed by large-scale analyses of human pluripotent stem cells. Proceedings of the National Academy of Sciences of the United States of America 110, 20569-20574.
Krey, J. F., Pasca, S. P., Shcheglovitov, A., Yazawa, M., Schwemberger, R., Rasmusson, R., and Dolmetsch, R. E. (2013). Timothy syndrome is associated with activity-dependent dendritic retraction in rodent and human neurons. Nature neuroscience 16, 201-209.
Laflamme, M. A., Chen, K. Y., Naumova, A. V., Muskheli, V., Fugate, J. A., Dupras, S. K., Reinecke, H., Xu, C., Hassanipour, M., Police, S., et al. (2007). Cardiomyocytes derived from human embryonic stem cells in pro-survival factors enhance function of infarcted rat hearts. Nature biotechnology 25, 1015-1024.
Laflamme, M. A., and Murry, C. E. (2011). Heart regeneration. Nature 473, 326-335.
Leyton-Mange, J. S., Mills, R. W., Macri, V. S., Jang, M. Y., Butte, F. N., Ellinor, P. T., and Milan, D. J. (2014). Rapid Cellular Phenotyping of Human Pluripotent Stem Cell-Derived Cardiomyocytes using a Genetically Encoded Fluorescent Voltage Sensor. Stem cell reports 2, 163-170.
Lian, X., Zhang, J., Azarin, S. M., Zhu, K., Hazeltine, L. B., Bao, X., Hsiao, C., Kamp, T. J., and Palecek, S. P. (2013). Directed cardiomyocyte differentiation from human pluripotent stem cells by modulating Wnt/beta-catenin signaling under fully defined conditions. Nature protocols 8, 162-175.
Ma, J., Guo, L., Fiene, S. J., Anson, B. D., Thomson, J. A., Kamp, T. J., Kolaja, K. L., Swanson, B. J., and January, C. T. (2011). High purity human-induced pluripotent stem cell-derived cardiomyocytes: electrophysiological properties of action potentials and ionic currents. American journal of physiology Heart and circulatory physiology 301, H2006-2017.
Minami, I., Yamada, K., Otsuji, T. G., Yamamoto, T., Shen, Y., Otsuka, S., Kadota, S., Morone, N., Barve, M., Asai, Y., et al. (2012). A small molecule that promotes cardiac differentiation of human pluripotent stem cells under defined, cytokine- and xeno-free conditions. Cell reports 2, 1448-1460.
Moretti, A., Bellin, M., Welling, A., Jung, C. B., Lam, J. T., Bott-Flugel, L., Dom, T., Goedel, A., Hohnke, C., Hofmann, F., et al. (2010). Patient-specific induced pluripotent stem-cell models for long-QT syndrome. The New England journal of medicine 363, 1397-1409.
Okita, K., Matsumura, Y., Sato, Y., Okada, A., Morizane, A., Okamoto, S., Hong, H., Nakagawa, M., Tanabe, K., Tezuka, K., et al. (2011). A more efficient method to generate integration-free human iPS cells. Nature methods 8, 409-412.
Pasca, S. P., Portmann, T., Voineagu, I., Yazawa, M., Shcheglovitov, A., Pasca, A. M., Cord, B., Palmer, T. D., Chikahisa, S., Nishino, S., et al. (2011). Using iPSC-derived neurons to uncover cellular phenotypes associated with Timothy syndrome. Nature medicine 17, 1657-1662.
Rais, Y., Zviran, A., Geula, S., Gafni, O., Chomsky, E., Viukov, S., Mansour, A. A., Caspi, I., Krupalnik, V., Zerbib, M., et al. (2013). Deterministic direct reprogramming of somatic cells to pluripotency. Nature 502, 65-70.
Shinagawa, T., Takagi, T., Tsukamoto, D., Tomaru, C., Huynh, L. M., Sivaraman, P., Kumarevel, T., Inoue, K., Nakato, R., Katou, Y., et al. (2014). Histone variants enriched in oocytes enhance reprogramming to induced pluripotent stem cells. Cell stem cell 14, 217-227.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Takahashi, K., Tanabe, K., Ohnuki, M., Narita, M., Sasaki, A., Yamamoto, M., Nakamura, M., Sutou, K., Osafune, K., and Yamanaka, S. (2014). Induction of pluripotency in human somatic cells via a transient state resembling primitive streak-like mesendoderm. Nature communications 5, 3678.
Warren, L., Manos, P.D., Ahfeldt, T., Loh, Y. H., Li, H., Lau, F., Ebina, W., Mandal, P. K., Smith, Z. D., Meissner, A., et al. (2010). Highly efficient reprogramming to pluripotency and directed differentiation of human cells with synthetic modified mRNA. Cell stem cell 7, 618-630.
Yazawa, M., Hsueh, B., Jia, X., Pasca, A. M., Bernstein, J. A., Hallmayer, J., and Dolmetsch, R. E. (2011). Using induced pluripotent stem cells to investigate cardiac phenotypes in Timothy syndrome. Nature 471, 230-234.
Yoshida, Y., Takahashi, K., Okita, K., Ichisaka, T., and Yamanaka, S. (2009). Hypoxia enhances the generation of induced pluripotent stem cells. Cell stem cell S, 237-241.
Yu, J., Hu, K., Smuga-Otto, K., Tian, S., Stewart, R., Slukvin, I I, and Thomson, J. A. (2009). Human induced pluripotent stem cells free of vector and transgene sequences. Science 324, 797-801.
Zhao, Y., Araki, S., Wu, J., Teramoto, T., Chang, Y. F., Nakano, M., Abdelfattah, A. S., Fujiwara, M., Ishihara, T., Nagai, T., et al. (2011). An expanded palette of genetically encoded Ca(2)(+) indicators. Science 333, 1888-1891.

### GENERAL METHODS

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

### INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims. The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

What is claimed is:

1. A composition comprising cardiac differentiated stem cells, wherein the cardiac differentiated stem cells are differentiated from pluripotent stem cells in the presence of one or more glycogen synthase kinase 3 (GSK3) signaling inhibitors and/or one or more WNT signaling inhibitors.

2. The composition of claim 1, wherein the one or more GSK3 signaling inhibitors comprise CHIR99021 (CHIR) and/or BIO (2′Z,3′E)-6-Bromoindirubin-3′-oxime).

3. The composition of claim 1, wherein the one or more WNT signaling inhibitors comprise IWP-3.

4. The composition of claim 1, wherein the pluripotent stem cells comprise induced pluripotent stem (iPS) cells.

5. The composition of claim 1, wherein the pluripotent stem cells are derived from healthy cells.

6. The composition of claim 1, wherein the pluripotent stem cells are derived from skin fibroblasts.

7. The composition of claim 1, wherein the pluripotent stem cells are derived from patient-derived cells.

8. The composition of claim 1, wherein the pluripotent stem cells are derived from cells from a patient with Timothy Syndrome.

9. The composition of claim 1, wherein the one or more GSK3 signaling inhibitors comprise CHIR having a concentration ranging from about 1 μM to about 5 μM.

10. The composition of claim 9, wherein CHIR has a concentration of about 3 μM.

11. The composition of claim 9, wherein CHIR has a concentration of about 5 μM.

12. The composition of claim 1, wherein the one or more GSK3 signaling inhibitors comprise BIO having a concentration of about 1 μM.

13. The composition of claim 1, wherein the one or more WNT signaling inhibitors comprise IWP-3 having a concentration of about 2 μM.
